(19) United States
(12) Patent Application Publication
(10) Pub. No.: US 2021/0251732 A1
(43) Pub. Date: Aug. 19, 2021

(54) HANDPIECES FOR MEDICAL AND DENTAL DEVICES

(71) Applicant: Parkell, Inc., Edgewood, NY (US)

(72) Inventor: Andres FRANCO, Islip, NY (US)

(21) Appl. No.: 17/176,588

(22) Filed: Feb. 16, 2021

Related U.S. Application Data
(60) Provisional application No. 62/976,470, filed on Feb. 14, 2020.

(51) Int. Cl. A61C 17/02; A61B 18/20; A61B 18/14; A61C 13/15; A61C 1/08; A61C 17/20; A61C 17/022

(52) U.S. Cl. CPC A61C 17/0202; A61B 18/20; A61B 18/14; A61B 2017/00424; A61C 1/087; A61C 17/20; A61C 17/022; A61C 19/004

(57) ABSTRACT

A handpiece of a medical or dental device allows a user to hold, support, and/or operate the handpiece by placing a portion of the user's hand, such as one or more fingers (e.g., the forefinger) through an ergonomic aperture in the handpiece.

### CROSS REFERENCE TO RELATED APPLICATION

This application is a continuation of U.S. Provisional Application Ser. No. 62/976,470, filed Feb. 14, 2020 the entire contents of which are incorporated herein by reference in its entirety.

### BACKGROUND

Conventional handpieces of medical and dental devices are typically elongated and cylindrical in shape and require practitioners or users to hold the handpieces, sometimes for prolonged periods, during clinical use. The process of holding such conventional handpieces requires, in some cases, the application or even sustained application by users of opposing finger forces or pressure (e.g., pinch force) between two or more portions of the practitioner's hand (e.g., two or more fingers), in order to hold, operate, manipulate, and effectively direct the handpiece (and rotational elements of the handpiece, in present, such as rotatable tips) during clinical use. Such manual or digital operation and/or manipulation and holding of such conventional handpieces has been found to cause repetitive stress injuries, such as for example, to the fingers, hand, wrist, and/or elbow of the user.

There is a need, therefore, for improved handpieces for medical and dental devices which lessen, alleviate, minimize, and/or eliminate required pinch forces associated with conventional handpieces. Moreover, there is a need for medical and dental devices which include ergonomic elements that address or minimize the occurrence and/or likelihood of repetitive stress injuries (which, if unaddressed, can cause pain, usage issues, and/or even career limitations for the practitioner).

### SUMMARY

In embodiments, in accordance with the principles of the present disclosure, a handpiece for a medical or dental device is provided and includes a proximal end portion, a distal end portion configured to be grasped between two or more fingers of a user, and an intermediate portion interconnecting the proximal and distal end portions. The intermediate portion defines an aperture therethrough configured for receipt of at least one of the two or more fingers of the user. The proximal end portion defines a first longitudinal axis, and the distal end portion defines a second longitudinal axis that is offset from and parallel with the first longitudinal axis of the proximal end portion.

In embodiments, the intermediate portion may include first and second split elements that define the aperture therebetween.

In embodiments, at least one of the first and second split elements may define a fluid channel therethrough.

In embodiments, the handpiece may further include a powder storage element in fluid communication with the fluid channel, and a proximal connector configured to receive a liquid, gas, or powder. The proximal connector may be in fluid communication with the fluid channel.

In embodiments, the handpiece may further include a nozzle extending distally from the distal end portion and in fluid communication with the fluid channel.

In embodiments, the intermediate portion may define a third longitudinal axis that is angled relative to the first and second longitudinal axes.

In embodiments, the intermediate portion may be linear along its length.

### BRIEF DESCRIPTION OF THE DRAWINGS

The present disclosure will become more readily apparent from the specific description accompanied by the following drawings, in which:

FIG. 1 is a top, perspective view illustrating an exemplary handpiece of the present disclosure;

FIG. 2 is a side view of the handpiece of FIG. 1;

FIG. 3 is a side view of the handpiece of FIG. 1 illustrating a hand of a user grasping the handpiece;

FIG. 4 is a perspective view illustrating another exemplary handpiece of the present disclosure;

FIG. 5 is a side view of the handpiece of FIG. 4;

FIG. 6 is a top, perspective view illustrating yet another exemplary handpiece of the present disclosure including an insert; and

FIG. 7 is a side view of the handpiece of FIG. 6.

### DETAILED DESCRIPTION

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior.”

As used herein, the term “distal” refers to the portion that is being described which is further from a user, while the term “proximal” refers to the portion that is being described which is closer to a user.

As used herein, the terms parallel and perpendicular are understood to include relative configurations that are substantially parallel and substantially perpendicular up to about +or −20 degrees from true parallel and true perpendicular.

The present disclosure relates to improved handpieces and devices for use in medical and/or dental applications. The handpieces described herein address the above-noted issues by incorporating an ergonomic element and/or aperture (e.g., an angled or sloped ergonomic aperture), which significantly lessen the hand pressure (e.g., pinch force) required for users to hold and operate the handpieces as compared to conventional handpieces, and which enable users to maintain a neutral finger, hand, and/or wrist position during use. The ergonomic element allows for the alleviation, minimization, or even elimination of repetitive stress injuries to the hand and/or wrist of medical or dental practitioners, particularly those associated with gripping (e.g., the use of pinch force to hold) conventional medical or dental handpieces during clinical use and those associated with non-neutral hand and/or wrist positioning during use (e.g., undesirable hand and/or wrist flexion, and undesirable hand and/or wrist extension). This is achieved by eliminating the need for practitioners to hold handpieces in the manner that is typical with conventional handpieces, such as via the use of opposing forces of the fingertips. The handpieces contemplated herein enable users to hold, support, and/or operate the handpiece by placing a portion of the user's hand, such as one or more fingers (e.g., the forefinger) through an ergonomic aperture in the handpiece.

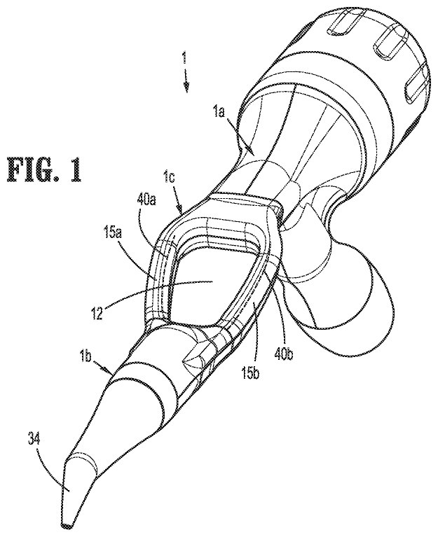

FIGS. 1-3 illustrate an exemplary handpiece (1) of a dental device and defines an ergonomic aperture (12) therethrough. A forefinger of a user's hand is placed through the ergonomic aperture (12) and at least a portion of the weight of the handpiece (1) is distributed atop a proximal portion “P” of the user's hand (e.g., a portion of the user's hand which is proximate to the third knuckle of the forefinger and/or back of the user's hand). In embodiments, substantially less weight and/or down-force associated with the handpiece (1) is experienced by the user at distal portions of their hand (e.g., fingertips), as compared to conventional handpieces. In embodiments, for example, minimal or no pinch-force is required by the user to support downforce and/or weight associated with distal portions of the handpiece (1). In embodiments, the user is therefore free (or substantially free) to use distal portions of their hand (for example, thumb and forefinger) to guide, operate, rotate, and/or manipulate a distal end portion (1b) of the handpiece (1) during clinical use.

The handpiece (1) of FIGS. 1-3 is a dental air polishing device, e.g., an air flow therapy device, or a powder flow therapy device. The handpiece (1) generally includes a proximal end portion (1a), the distal end portion (1b), and an intermediate portion (1c) interconnecting the proximal and distal end portions (1a), (1b). The proximal end portion (1a) and the distal end portion (1b) may each define discrete longitudinal axes (22), (24) that are offset from and parallel with one another. The intermediate portion (1c) defines the ergonomic aperture (12) therethrough. The intermediate portion (1c) defines a longitudinal axis (23) that extends at a non-parallel angle relative to the longitudinal axes (22), (24) of the proximal and distal end portions (1a), (1b). In embodiments, the longitudinal axis (23) of the intermediate portion (1c) may be an obtuse angle (e.g., from about 100 degrees to about 170 degrees), an acute angle (e.g., from 10 degrees to about 80 degrees), or a right angle (e.g., about 90 degrees) relative to the longitudinal axes (22), (24) of the proximal and distal end portions (1a), (1b). In embodiments, the intermediate portion (1c) may extend at about 150 degrees from the proximal end portion (1a) and about 150 degrees from the distal end portion (1b). In embodiments, the intermediate portion (1c) may be linear along its length. In embodiments, the intermediate portion (1c) may be curved along its length, bent at various points along its length, or assume any suitable configuration that positions the distal end portion (1b) of the handpiece (1) on a different plane from the proximal end portion (1a).

The intermediate portion (1c) includes a split portion (e.g., a sloped split portion or element) that has split elements (15a and 15b), such as, for example, elongated hollow arms that cooperatively define the ergonomic aperture (12). In this regard, in embodiments, the ergonomic aperture (12) is sloped or angled downward relative to the proximal end portion (1a). Each of the split elements (15a and 15b) defines a channel or conduit (40a and 40b) (shown in phantom) therethrough for transferring fluids therethrough (e.g., gases, liquids, powders). The channels (40a and 40b) each terminate distally at the nozzle (34) from which the conveyed fluids are discharged out of the handpiece (1). In embodiments, the channels (40a and 40b) may accommodate a tube therein.

The proximal end portion (1a) may include a powder storage element (26) in which air mixes with stored powder(s), a stem element (28), and a proximal connector element (30) for optionally connecting to conventional air and water sources. In embodiments, the powder storage element (26) may be in fluid communication with the channel (40a) of split element (15a), whereas a water line originating from the proximal connector element (30) may be in fluid communication with the channel (40b) of split element (15b). In embodiments, the powder storage element (26) may be in fluid communication with the channel (40b) of split element (15b), whereas a water line originating from the proximal connector element (30) may be in fluid communication with the channel (40a) of split element (15a). In embodiments, the powder storage element (26) may be in fluid communication with each of the channels (40a and 40b) of the split elements (15a and 15b). In embodiments, the powder storage element (26) may be in fluid communication, e.g., via a dedicated conduit, along with a separate water line, both contained within either split element (15a) or 15(b).

Any suitable coupling element can be used in the context of the present disclosure for removably attaching the handpiece (1) to standard air/water supply lines in dental operatories. Moreover, one of the channels may be for water and the other one for air. The air conduit enters and releases air into the powder storage element (26) and the same or a different conduit collects an air-powder mixture (wherein, in some embodiments, holes or apertures present in the exist conduit of a size to permit only powder particles of a specific size range to enter and thus depart the powder storage element within the egressing air-powder conduit).

Any suitable mixing element can be used in the context of the present disclosure, for mixing air with powder such as, for example, as described in U.S. Pat. No. 9,974,629, U.S. Pat. No. 8,152,524, U.S. Pat. No. 6,719,561, U.S. Pat. No. 5,857,851, the contents of which are herein incorporated by reference in their entirety, or similar elements for adequately mixing air and powder. In this regard, the device can be used with any suitable powder that's suitable for subgingival and/or supragingival use, such as, for example, sodium bicarbonate, glycine, erythritol, or a combination or mixture thereof.

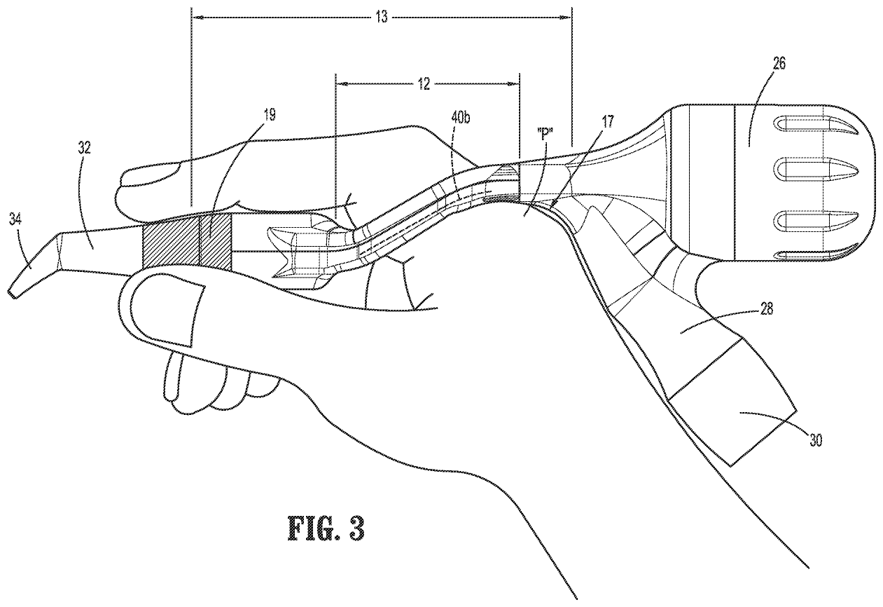

As is depicted in FIG. 3 for example, the distal end portion (1b) of the handpiece (1) may include a distal housing (32) extending from the intermediate portion (1c) and a distal engagement element (19) disposed about the distal housing (32). In embodiments, the distal engagement element (19) is fabricated from a friction-enhancing material, such as, for example, rubber or the like. The distal engagement element (19) may extend only partially along the length of the distal end portion (1b) or along the entire length of the distal end portion (1b). The distal engagement element (19) is located a selected distance from the aperture (12) so that a tip of a user's forefinger will be guided to rest thereon. The distal end portion (1b) may have a distal tip, such as, for example, a spray nozzle (34) that extends distally and at a downward angle from the distal housing (32) between about 90 degrees and about 150 degrees, e.g., about 90 degrees, about 91 degrees, about 92 degrees, about 93 degrees, about 94 degrees, about 95 degrees, about 96 degrees, about 97 degrees, about 98 degrees, about 99 degrees, about 100 degrees, about 101 degrees, about 102 degrees, about 103 degrees, about 104 degrees, about 105 degrees, about 106 degrees, about 107 degrees, about 108 degrees, about 109 degrees, about 110 degrees, about 111 degrees, about 112 degrees, about 113 degrees, about 114 degrees, about 115 degrees about 116 degrees, about 117 degrees, about 118 degrees, about 119 degrees, about 120 degrees, about 121 degrees, about 122 degrees, about 123 degrees, about 124 degrees, about 125 degrees, about 126 degrees, about 127 degrees, about 128 degrees, about 129 degrees, about 130 degrees, about 131 degrees, about 132 degrees, about 133 degrees, about 134 degrees, about 135 degrees about 136 degrees, about 137 degrees, about 138 degrees, about 139 degrees, about 140 degrees, about 141 degrees, about 142 degrees, about 143 degrees, about 144 degrees, about 145 degrees, about 146 degrees, about 147 degrees, about 148 degrees, about 149 degrees, about 150 degrees. In embodiments, the spray nozzle (34) may extend linearly with the distal housing (32). In embodiments, the spray nozzle (34) is in fluid communication with terminal distal ends of each of the channels (40a and 40b) for mixing and dispensing an air powder stream flowing from the first channel (40a) and a water stream (or another suitable liquid) flowing from the second channel (40b).

Any suitable one or more valve element(s) and nozzle element(s) can be used in the context of the present disclosure, such as for example, as described in U.S. patent application 2016/0270889 and U.S. Pat. Nos. 6,149,509 and 9,974,629, the contents of which are herein incorporated by reference in their entirety.

Any suitable patient-contacting tips that are removable or disconnectable from the handpiece (1) can be used in the context of the present disclosure, such as for example, any suitable removable sub-gingival tip and/or any suitable supra-gingival tip, or the like. In embodiments, the tips can be made of any suitable material, such as for example, any suitable autoclaveable material. The tips can be reusable autoclaveable tips (e.g., subgingival and/or supragingival) or even single-use or single-patient-use tips.

In embodiments, the handpiece (1) includes an ergonomic element (13) which extends from (i) the portion of the distal engagement element (19) where the user's fingers make contact (e.g., the center of the distal engagement element, as measured from distal to proximal ends) to (ii) a point proximate to where the stem axis (30) and upper longitudinal axis (24) intersect. Moreover, the handpiece may include a rest plate (17) under which a portion of the user's hand (e.g., a portion of the user's forefinger, such as for example the third knuckle of the forefinger) is situated. In embodiments, the rest plate may have an arcuate shape dimensioned to accommodate the back portion “P” of the user's hand when the user's forefinger and thumb are in a grasping state.

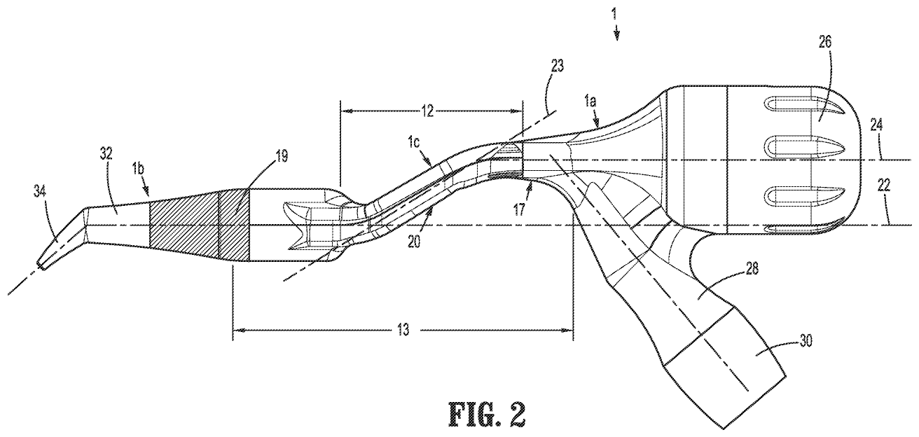

In embodiments, e.g., (as is depicted in FIG. 2, for example), the ergonomic element (13) extends from (i) a portion of the handpiece (1) which is proximate to the intersection of a stem axis (30) and upper longitudinal axis (24), and (ii) a portion of the distal engagement element (19).

The term “ergonomic aperture length” is defined herein to mean the distal to proximal length of the aperture hole (12). The terms “ergonomic element” and “ergonomic element length” are defined herein to refer to the element, and the length thereof, which are defined by distance between (i) the point or area where one or more of the user's fingertips engage the distal housing or distal end portion (1a) of the handpiece when in use, and (ii) a portion of the handpiece, along the upper longitudinal axis (24), which is proximal the ergonomic aperture (12) (e.g., in embodiments where the handpiece is a dental air polishing device, intersection point of the stem axis and upper longitudinal axis as described herein).

The ergonomic element (13) and aperture (12) can be configured in any suitable manner, such as, in any suitable position on the handpiece, orientation with respect to the handpiece, as well as any ergonomically-suitable size and shape. In addition, the ergonomic element, ergonomic aperture, and split elements can have any ergonomically-suitable angle or slope or contour relative to a lower longitudinal axis (22) and upper longitudinal axis (24) of the handpiece. In this regard, it has been surprisingly discovered, in embodiments, that the combination of (ii) the sloped ergonomic element with (ii) the proper length of the ergonomic element (13) leads to optimum ergonomic positioning of the user's fingers, hand, and wrist (e.g., at neutral hand and neutral wrist position) during handpiece usage (such as hand and wrist postures which are lessen the risk of repetitive stress injuries).

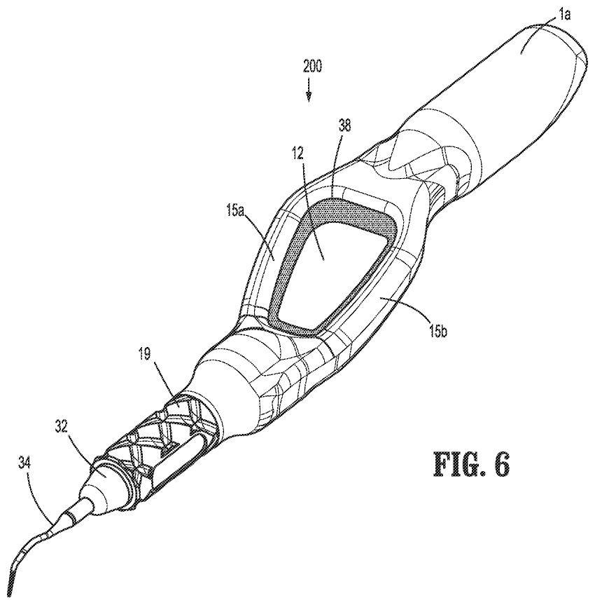

In embodiments, the handpieces may further include one or more insert elements 38 (FIG. 6), such as elements including plastic, rubber, silicone or the like, and preferably steam-sterilizable or autoclaveable. The insert elements 38 are configured in size and shape to mate and/or be inserted into the ergonomic aperture, in such a manner so that the insert element 38 is removably placed into the aperture and makes the aperture size-adaptable and/or comfort-adaptable for the user of the device. In embodiments, the device includes two or more insert elements 38 of differing size, so that users can choose and removably-insert that specific insert element 38 (into the ergonomic aperture) which renders the device and ergonomic aperture most comfortable and/or functional for that specific user. In embodiments, the insert element 38 is removed and autoclaved (or disposed of) between distinct patients.

In those embodiments in which the distal end portion (1a) of the handpiece (1) is rotatable, such rotation can be controlled by the user via any suitable rotation element. In embodiments, the distal end portion (1a) is rotatable by the user through use of the user's thumb and opposing finger. In embodiments, the rotation is digitally, electrically, electronically, or even mechanically controlled by the used through engagement or pushing of a rotation control element.

In embodiments, it was discovered that by incorporating a “sloped or angled ergonomic aperture” into the handpiece, the ergonomic stresses experienced by the hand, wrist, arm, and/or elbow of the user (or the like) are significantly reduced as compared to stresses encountered with conventional devices not having a sloped or angled ergonomic aperture. For ease of reference, when embodiments of the present device include a sloped or angled ergonomic aperture, the slope or angle of the ergonomic aperture at a position between the distal and proximal ends of the device cause the device to not have one single longitudinal axis; rather, as is depicted in FIG. 2, the device having a sloped ergonomic aperture further includes the lower longitudinal axis (22) and the upper longitudinal axis (24), wherein the sloped ergonomic aperture (12) is situated and/or oriented such that it forms an “aperture angle” with the lower and upper longitudinal axes (22), (24).

It was surprisingly and unexpectedly discovered in embodiments, that the combination of the ergonomic angle and the ergonomic aperture length and the ergonomic element length are critical for maximizing an “ergonomic effect for users” (meaning, the greatest reduction in hand, wrist, elbow pressures and stresses, and the like). In embodiments, the handpiece includes an aperture angle of about 20 to about 40 degrees relative to the lower longitudinal axis. In embodiments, the device includes an aperture angle of about 25 to about 35 degrees relative to the lower longitudinal axis. In embodiments, the device includes an aperture angle of about 28 to about 32 degrees relative to the lower longitudinal axis.

In embodiments, the device includes an ergonomic aperture length of about 0.5 to about 3 inches. In embodiments, the device includes an ergonomic aperture length of about 0.75 to about 2.5 inches. In embodiments, the device includes an ergonomic aperture length of about 0.75 to about 2 inches. In embodiments, the device has an ergonomic aperture length of about 0.75 to about 1.5 inches. In embodiments, the device includes an ergonomic aperture length of about 0.75 to about 1.25 inches. In embodiments, the device has an ergonomic aperture length of about 1 to about 1.5 inches. In embodiments, the device has an ergonomic aperture length of about 1 to about 1.25 inches.

In embodiments, the device has an ergonomic element length of about 2.5 to about 4.5 inches. In embodiments, the device has an ergonomic element length of about 3 to about 4 inches. In embodiments, the device has an ergonomic element length of about 2.75 to about 3.75 inches. In embodiments, the device includes an ergonomic element length of about 2.5 to about 3.5 inches. In embodiments, the device has an ergonomic element length of about 2.25 to about 3 inches. In embodiments, the device includes an ergonomic element length of about 2.5 to about 3 inches.

In embodiments, the device includes (i) an ergonomic angle of about 20 to about 40 degrees (e.g., about 25 to about 35 degrees, or about 28 to about 32 degrees) relative to the lower longitudinal axis (22), and (ii) an ergonomic aperture length of about 0.5 to about 3 inches (e.g., about 0.75 to about 2.5 inches, about 0.75 to about 2 inches, about 0.75 to about 1.5 inches, about 0.75 to about 1.25 inches, about 1 to about 1.5 inches, or about 1 to about 1.25 inches).

In embodiments, the device includes (i) an ergonomic angle of about 20 to about 40 degrees (e.g., about 25 to about 35 degrees, or about 28 to about 32 degrees) relative to the lower longitudinal axis, and (ii) an ergonomic element length of about 2.5 to about 4.5 inches (e.g., about 3 to about 4 inches, about 2.75 to about 3.75 inches, about 2.5 to about 3.5 inches, about 2.25 to about 3 inches, or about 2.5 to about 3 inches).

In embodiments, the device includes (i) an ergonomic aperture length of about 0.5 to about 3 inches (e.g., about 0.75 to about 2.5 inches, about 0.75 to about 2 inches, about 0.75 to about 1.5 inches, about 0.75 to about 1.25 inches, about 1 to about 1.5 inches, or about 1 to about 1.25 inches), and (ii) an ergonomic element length of about 2.5 to about 4.5 inches (e.g., about 3 to about 4 inches, about 2.75 to about 3.75 inches, about 2.5 to about 3.5 inches, about 2.25 to about 3 inches, or about 2.5 to about 3 inches).

In embodiments, the device includes (i) an ergonomic angle of about 20 to about 40 degrees (e.g., about 25 to about 35 degrees, or about 28 to about 32 degrees) relative to the lower longitudinal axis, (ii) an ergonomic aperture length of about 0.5 to about 3 inches (e.g., about 0.75 to about 2.5 inches, about 0.75 to about 2 inches, about 0.75 to about 1.5 inches, about 0.75 to about 1.25 inches, about 1 to about 1.5 inches, or about 1 to about 1.25 inches), and (iii) an ergonomic element length of about 2.5 to about 4.5 inches (e.g., about 3 to about 4 inches, about 2.75 to about 3.75 inches, about 2.5 to about 3.5 inches, about 2.25 to about 3 inches, or about 2.5 to about 3 inches).

For purposes of cleaning and/or maintenance, the handpiece (1) may be disassembleable at any desired points for purposes of accessing and cleaning the internal channels or cores of the water channel and the air/powder channel (40a and 40b), such as for example, at the midpoint of distal or proximal ends of elements (15a) and (15b), or at any desired points distal or proximal to elements (15a) and (15b). In embodiments, the interior/internal channels or cores of the water conduit and air/powder conduit (40a and 40b) are cleaned, evacuated, and/or cleared by applying a source of pressurized air flow (even, e.g., a rubber bulb which is fitted to the outer diameter of the distal end of the handpiece), and which when engaged causes high-pressure air to travel through the conduits in a distal to proximal direction.

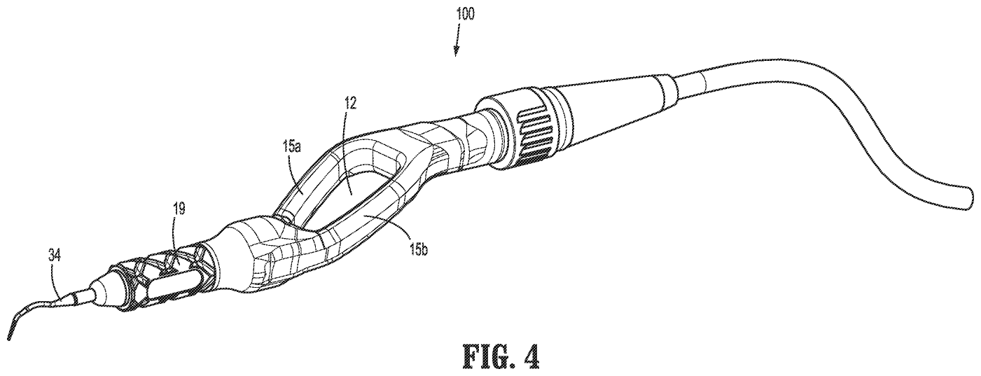

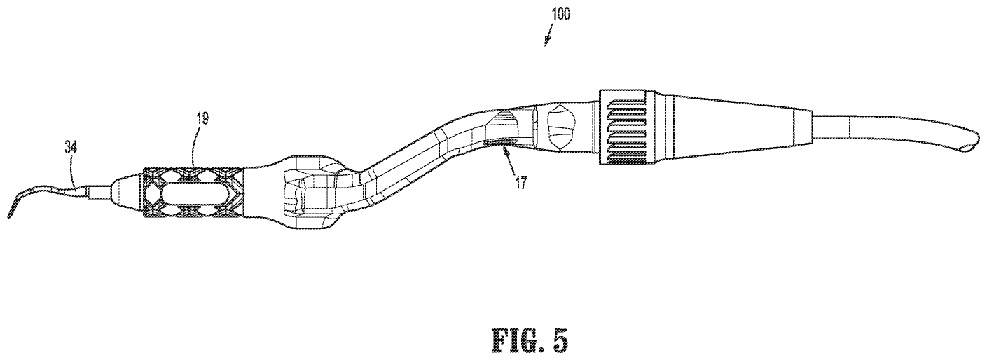

FIGS. 4-5 depict embodiments wherein the handpiece (100) is a corded medical or dental device (e.g., a magnetostrictive ultrasonic scaler, a piezoelectric ultrasonic scaler, or electrosurgical device) which includes an ergonomic element and aperture (e.g., a sloped or angled ergonomic element and aperture) as described herein.

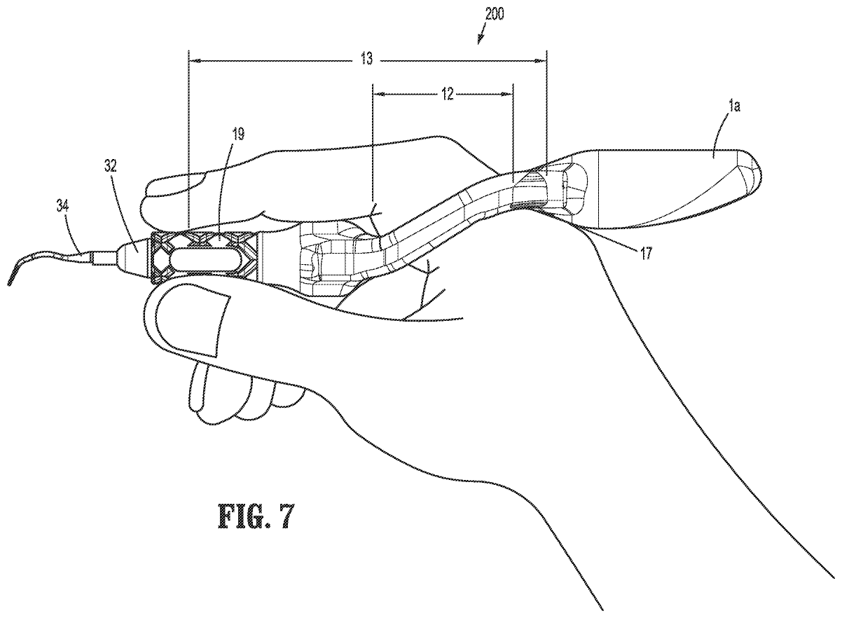

FIGS. 6-7 depict embodiments wherein the handpiece (200) is a cordless medical or dental device (e.g., a dental curing light, electro surgical device, or surgical laser) which includes an ergonomic element and aperture (e.g., a sloped or angled ergonomic element and aperture) as described herein.

In embodiments, the handpiece or device is autoclaveable. In this regard, for example, the handpiece or device may be composed of materials (such as metal, suitable plastics, and/or the like, or combinations or mixtures thereof) which render the handpiece autoclaveable for any desired number of autoclaving cycles, such as for example, 1000 autoclaving cycles, 1500 autoclaving cycles, 2000 autoclaving cycles, 3000 autoclaving cycles, or even 5000 or 10,000 autoclaving cycles.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A handpiece of a medical or dental device, comprising:
a proximal end portion defining a first longitudinal axis;
a distal end portion configured to be grasped between two or more fingers of a user, the distal end portion defining a second longitudinal axis that is offset from and parallel with the first longitudinal axis of the proximal end portion; and
an intermediate portion interconnecting the proximal and distal end portions, wherein the intermediate portion defines an aperture therethrough configured for receipt of at least one of the two or more fingers of the user.

2. The handpiece according to claim 1, wherein the intermediate portion includes first and second split elements that define the aperture therebetween.

3. The handpiece according to claim 2, wherein at least one of the first and second split elements defines a fluid channel therethrough.

4. The handpiece according to claim 3, further comprising:
a powder storage element coupled to the proximal end portion and in fluid communication with the fluid channel; and
a proximal connector coupled to the proximal end portion and configured to receive a liquid, gas, or powder, the proximal connector being in fluid communication with the fluid channel.

5. The handpiece according to claim 4, further comprising a nozzle extending distally from the distal end portion and in fluid communication with the channel.

6. The handpiece according to claim 1, wherein the intermediate portion defines a third longitudinal axis that is angled relative to the first and second longitudinal axes.

7. The handpiece according to claim 6, wherein the intermediate portion is linear along its length.

8. The handpiece according to claim 1, wherein the handpiece is selected from the group consisting of a magnetostrictive ultrasonic scaler, a piezoelectric ultrasonic scaler, or an electrosurgical device.

9. The handpiece according to claim 1, wherein the handpiece is selected from the group consisting of a dental curing light, an electrosurgical device, or a surgical laser.
